(19) United States
(12) Patent Application Publication
(10) Pub. No.: US 2006/0058548 A1
(43) Pub. Date: Mar. 16, 2006

(54) Process of preparing O-carbamoyl compounds in the presence of active amine group

(75) Inventors: Yong Moon Choi, Pine Brook, NJ (US); Min Woo Kim, Montville, NJ (US)

Correspondence Address: GIBBONS, DEL DEO, DOLAN, GRIFFINGER & VECCHIONE, 1 RIVERFRONT PLAZA, NEWARK, NJ, 07102-5497, US

(73) Assignee: SK Corporation

(21) Appl. No.: 11/266,555

(22) Filed: Nov. 3, 2005

Related U.S. Application Data
(63) Continuation of application No. 10/680,979, filed on Oct. 8, 2003, now abandoned.

(51) Int. Cl. C07D 217/12

(52) U.S. Cl. 560/157; 546/146

(57) ABSTRACT

A process for preparing O-carbamoyl aminoalcohols represented by Formula I 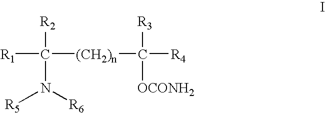 wherein: n is an integer from 0 and 5;
R₁, R₂, R₃ and R₄ are individually selected from the group consisting of hydrogen, alkyl, cycloalkyl, substituted or unsubstituted aryl and arylalkyl the aryl portion of which may be unsubstituted or substituted:
R₅ and R₆ are individually selected from the group consisting of hydrogen, alkyl or arylalkyl the aryl portion of which may be unsubstituted or substituted; or
R₁ and R₅ together with the carbon and nitrogen to which they are attached may form an unfused or fused heterocyclic ring having from 4 to 10 members.
   comprising reacting an aminoalcohol represented by Formula II 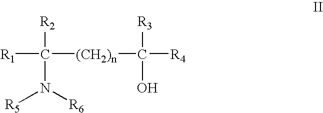
   wherein n, R₁, R₂, R₃, R₄, R₅ and R₆ are as defined; with a cyanate and an excess of an acid in an organic solvent medium.

### FIELD OF INVENTION

The present invention relates to a novel process for preparing O-carbamoyl aminoalcohols.

### BACKGROUND OF THE INVENTION

O-carbamoyl aminoalcohols comprise a new class of pharmaceutically useful compounds. For instance. O-carbamoyl-(D)-phenylalaninol hydrochloride and O-carbamoyl-(L)-3-hydroxymethyl-1,2,3,4-tetrahydroisoquinoline hydrochloride are being developed for the treatment of central nervous system (CNS) disorders, particularly as antidepressants.

Due to the generally higher reactivity of amines in comparison to hydroxyl groups, when the O-carbamoylated product of an aminoalcohol is synthesized, the amine moieties need to be protected prior to the carbamoylation reaction. Hence, a lengthy sequence of (1) protection. (2) carbamoylation reaction and (3) deprotection is typically required for the transformation as described in Scheme 1.

An example of the reaction in accordance with Scheme 1 would be the reaction of an aminoalcohol with benzyl chloroformate to form the protected N-benzyloxycarbonyl aminoalcohol. Carbamoylation of this protected aminoalcohol with phosgene followed by reaction with an amine yields the O-carbamoyl-N-protected aminoalcohol. The deprotection of this N-protected compound is achieved by hydrogenation. 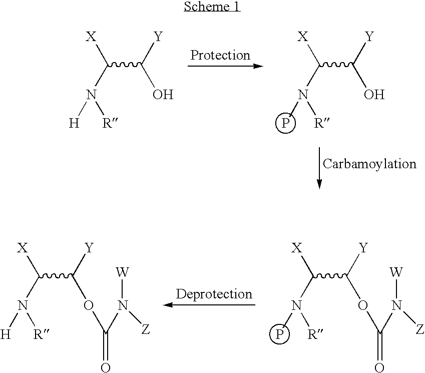
wherein W, X, Y and Z are individually selected from the group consisting of hydrogen, alkyl, cycloalkyl, aryl or arylalkyl; and,
R″ is selected from the group consisting of hydrogen, alkyl or arylalkyl.

This process has been advantageously simplified in accordance with the present invention.

### SUMMARY OF THE INVENTION

The present invention provides a novel process for preparing O-carbamoyl aminoalcohols via chemoselective carbamoylation of hydroxyl groups therein in a single step using a cyanate and an excess of acid in an organic medium. Particularly, the present invention involves the use of sodium cyanate and methanesulfonic acid in the single step preparation of O-carbamoyl aminoalcohols. Both small-scale laboratory preparations and large-scale industrial preparations are disclosed. The process is particularly advantageous for the preparation of O-carbamoyl-D-phenylalaninol, O-carbamoyl-(L)-oxymethyl-1,2,3,4-tetrahydroisoquinoline, and carbamic acid 2-((4-fluorobenzoyl)piperidin-1-yl)-1-phenylethyl ester.

### DETAILED DESCRIPTION OF THE INVENTION

The present invention provides a novel process for preparing O-carbamoyl aminoalcohols. The process is more efficient in introducing the carbamoyl moiety into the starting aminoalcohol than that previously known which is shown above in Scheme 1. As such, the present invention can be illustrated by Scheme 2: 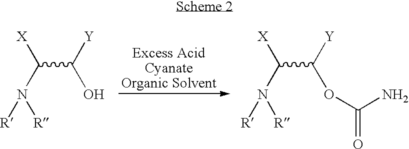
wherein
X and Y are individually selected from the group consisting of hydrogen, alkyl, cycloalkyl, aryl or arylalkyl: wherein the aryl portion may be substituted or unsubstituted by (X′)ₘ as defined below; and,
R′ and R″ are selected from the group consisting of hydrogen, alkyl or arylalkyl, wherein the aryl portion may be substituted or unsubstituted by (X′)ₘ as defined below.

It is quite surprising that the process described in the present invention, which employs an organic solvent system as the reaction medium, selectively produces the O-carbamoylated species as the dominant product. It should be noted that the reaction of aminoalcohols in aqueous acidic medium with a cyanate produces the N-carbamoylated product as the major product.

The present invention provides a novel process that is particularly advantageous for the preparation of O-carbamoyl aminoalcohols represented by Formula I 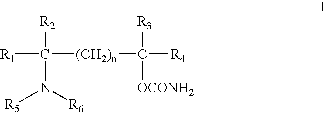
wherein:
n is an integer from 0 to 5;
R₁, R₂, R₃ and R₄ are individually selected from the group consisting of hydrogen, alkyl, cycloalkyl, substituted or unsubstituted aryl and arylalkyl wherein the aryl portion may be unsubstituted or substituted by (X′)ₘ, wherein m is an integer from 0 to 4 and X′ is selected from the group consisting of hydrogen, alkyl, alkoxy, alkylthio, halogen, hydroxy, nitro and trifluoromethyl;
R₅ and R₆ are individually selected from the group consisting of hydrogen, alkyl or arylalkyl wherein the aryl portion may be substituted or unsubstituted by (X′)ₘ, wherein m and X′ are as defined; or
R₁ and R₅ together with the carbon and nitrogen to which they are attached form an unfused or fused heterocyclic ring having from 4 to 10 members.
   The process comprises reacting an aminoalcohol represented by Formula II 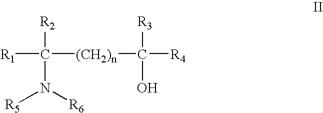
   wherein R₁ through R₆ and n are as defined above, with a cyanate and an excess of acid, in an organic solvent medium.

The starting aminoalcohol represented by the general structural Formula II may be chiral or achiral. The process described in the present invention can be used to prepare both the racemate and optically active forms of the desired O-carbamoyl aminoalcohol.

While specific reaction conditions may vary for individual starting aminoalcohol, the following description is of general conditions for the preparatory process of the present invention.

In accordance with the present invention, an excess of the acid is required for the protonation of the amine moieties present in the starting alcohol prior to the desired reaction. Typically, the amount of the acid is between about one and about ten molar equivalents in excess of amount required to react with the total number of amine groups present in the starting aminoalcohol represented by formula II. Hence, if one amine group is present, about two to about eleven equivalents of an acid are typically used, however, the presence of additional equivalents of acid does not hinder the reaction.

The acid utilized in the process of the present invention can be an organic or inorganic acid such as, for example, hydrochloric acid, sulfuric acid, phosphoric acid, acetic acid, halogenated acetic acids, arylsulfonic acids, alkylsulfonic acids and halogenated alkylsulfonic acids. Hydrochloric acid, halogenated acetic acids, arylsulfonic acids and alkylsulfonic acids are preferred for the subject synthesis. Particularly preferred acids include hydrochloric acid, trifluoroacetic acid, trichloroacetic acid, benzenesulfonic acid, toluenesulfonic acid, methanesulfonic acid, ethanesulfonic acid, and trifluoromethanesulfonic acid.

The present invention utilizes a cyanate to produce a cyanic acid in situ. Typically, the cyanate is used in about one to about ten mole equivalents of the starting aminoalcohol for the present invention. Useful cyanates for the present invention include, but are not limited to, alkali metal cyanates, such as sodium cyanate, potassium cyanate, and ammonium cyanate, alkaline earth cyanates, such as magnesium cyanate, calcium cyanate, and the like. Alternatively, rather than producing cyanic acid from a cyanate, purified cyanic acid may be employed which would also produce the desired product.

The carbamation reaction described in the present invention can be executed in various organic solvents. Halogenated alkanes such as dichloromethane; etheral solvents, such as tetrahydrofuran; nitrile solvents, such as acetonitrile; and aromatic solvents, such as toluene; or mixtures thereof can be used as the reaction solvent. Preferred solvents are selected from the group consisting of dichloromethane, chloroform, 1,2-dichloroethane, 1,1,1-trichloroethane, tetrahydrofuran, 1.2-dimethoxyethane, diethyl ether, acetonitrile, propionitrile, benzene, toluene, xylene and mixtures thereof. Halogenated alkanes and nitrile solvents including dichloromethane. 1,2-dichloroethane, 1,1,1-trichloroethane and acetonitrile are particularly preferred solvents.

The weight to volume ratio for the amount of the aminoalcohol represented by Formula II to the amount of the organic solvent medium is within the range from about 1:3 to about 1:100. For example, when one gram of aminoalcohol is employed, between about three and about one hundred milliliters of solvent would be utilized for the reaction.

The subject reaction is carried out at a temperature ranging from about −80° to about 80° C. depending upon the solvent employed. Typically, the reaction is carried out at temperatures ranging from about −10° C. to about 60° C. The reaction temperature will vary within the ranges given depending on the starting aminoalcohol.

In a typical reaction in accordance with the present invention, the starting aminoalcohol is placed in a reaction vessel followed by addition of the reaction solvent. The order of subsequent addition of the cyanate and the acid employed typically does not produce any significantly different result. Preferably, the reagent addition steps are carried out at temperatures ranging from about −10° C. to about 5° C.

A preferred embodiment of this invention provides a novel process for preparing O-carbamoyl aminoalcohol represented by Formula III 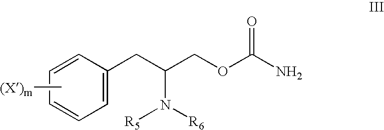
wherein X′, m, R₅ and R₆ are as defined.
The process comprises reacting an aminoalcohol represented by Formula IV 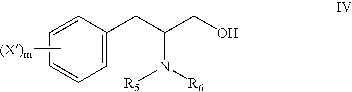
wherein X′, m, R₅ and R₆ are as defined;
with a cyanate selected from the group consisting of sodium cyanate, potassium cyanate, ammonium cyanate, magnesium cyanate, and calcium cyanate;
and an excess of an acid selected from the group consisting of hydrochloric acid, acetic acid, trifluoroacetic acid, trichloroacetic acid, benzenesulfonic acid, toluenesulfonic acid, methanesulfonic acid, ethanesulfonic acid, and trifluoromethanesulfonic acid;
in an organic solvent medium selected from the group consisting of dichloromethane, chloroform, 1.2-dichloroethane. 1.1.1-trichloroethane, tetrahydrofuran. 1.2-dimethoxyethane, diethyl ether, acetonitrile, propionitrile, benzene, toluene, xylene, and mixtures thereof.

Another preferred embodiment of this invention provides a novel process for preparing an O-carbamoyl aminoalcohol represented by Formula V 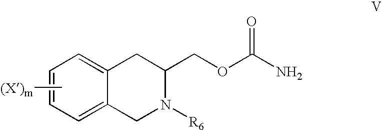
wherein X′, m, and R₆ are as defined.
The process comprises reacting an aminoalcohol represented by Formula VI 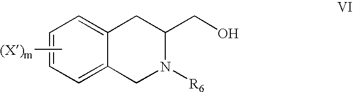
wherein X′, m, and R₆ are as defined;
with a cyanate selected from the group consisting of sodium cyanate, potassium cyanate, ammonium cyanate, magnesium cyanate, and calcium cyanate;
and an excess of an acid selected from the group consisting of hydrochloric acid, acetic acid, trifluoroacetic acid, trichloroacetic acid, benzenesulfonic acid, toluenesulfonic acid, methanesulfonic acid, ethanesulfonic acid, and trifluoromethanesulfonic acid;
in an organic solvent medium selected from a group consisting of dichloromethane, chloroform, 1.2-dichloroethane, 1.1.1-trichloroethane, tetrahydrofuran, 1.2-dimethoxyethane, diethyl ether, acetonitrile, propionitrile, benzene, toluene, xylene, and mixtures thereof.

Still another preferred embodiment of the present invention provides a novel process for preparing O-carbamoyl-D-phenylalaninol represented by Formula VII 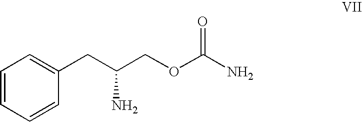
which comprises reacting D-phenylalaninol represented by Formula VIII 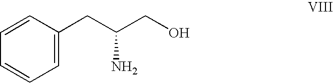
with a cyanate selected from the group consisting of sodium cyanate, potassium cyanate, ammonium cyanate, magnesium cyanate, and calcium cyanate;
and an excess of an acid selected from the group consisting of hydrochloric acid, acetic acid, trifluoroacetic acid, trichloroacetic acid, benzenesulfonic acid, toluenesulfonic acid, methanesulfonic acid, ethanesulfonic acid, and trifluoromethanesulfonic acid;
in an organic solvent medium selected from the group consisting of dichloromethane, chloroform, 1,2-dichloroethane, 1.1.1-trichloroethane, tetrahydrofuran, 1.2-dimethoxyethane, diethyl ether, acetonitrile, propionitrile, benzene, toluene, xylene, and mixtures thereof.

Still another preferred embodiment of the present invention provides a novel process for preparing O-carbamoyl-(L)-oxymethyl-1.2.3.4-tetrahydroisoquinoline represented by Formula IX 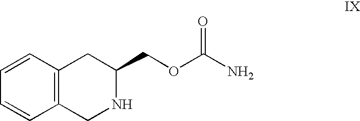
which comprises reacting (L)-3-hydroxymethyl-1.2.3.4-tetrahydroisoquinoline represented by Formula X 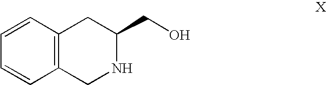
with a cyanate selected from the group consisting of sodium cyanate, potassium cyanate, ammonium cyanate, magnesium cyanate, and calcium cyanate;
and an excess of an acid selected from the group consisting of hydrochloric acid, acetic acid, trifluoroacetic acid, trichloroacetic acid, benzenesulfonic acid, toluenesulfonic acid, methanesulfonic acid, ethanesulfonic acid, and trifluoromethanesulfonic acid;
in an organic solvent medium selected from a group consisting of dichloromethane, chloroform, 1,2-dichloroethane, 1.1-trichloroethane, tetrahydrofuran, 1.2-dimethoxyethane, diethyl ether, acetonitrile, propionitrile, benzene, toluene, xylene and mixtures thereof.

Yet still another embodiment of the present invention provides a novel process for preparing carbamic acid 2-((4-fluorobenzoyl)piperidin-1-yl)-1-phenylethyl ester represented by Formula XI: 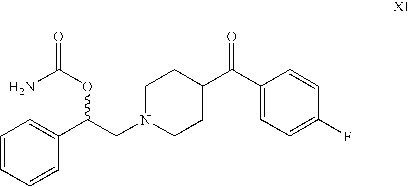
which comprises reacting 2-(4-fluorobenzoyl)piperidin-1-yl)-1-phenylethanol represented by Formula XII 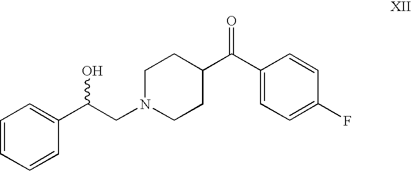
with a cyanate selected from the group consisting of sodium cyanate, potassium cyanate, ammonium cyanate, magnesium cyanate, and calcium cyanate;
and an excess of an acid selected from the group consisting of hydrochloric acid, acetic acid, trifluoroacetic acid, trichloroacetic acid, benzenesulfonic acid, toluenesulfonic acid, methanesulfonic acid, ethanesulfonic acid, and trifluoromethanesulfonic acid;
in an organic solvent medium selected from a group consisting of dichloromethane, chloroform, 1.2-dichloroethane, 1.1.1-trichloroethane, tetrahydrofuran, 1.2-dimethoxyethane, diethyl ether, acetonitrile, propionitrile, benzene, toluene, xylene and mixtures thereof.

Set forth below are definitions of the radicals covered by Formulae I to VI. As utilized herein, the term “alkyl” means a straight- or branched-chain hydrocarbon radical having from one to eight carbon atoms and includes, but is not limited to, methyl, ethyl, n-propyl, isopropyl, n-butyl, sec-butyl, isobutyl, tert-butyl, n-pentyl, n-hexyl, and the like, except where specifically stated otherwise.

The term “halogen” includes fluorine, chlorine, bromine, and iodine with fluorine and chlorine being preferred.

The term “alkoxy” refers to an alkyl radical attached to the remainder of the molecule by oxygen; this includes, but is not limited to, methoxy, ethoxy, and propoxy groups.

The term “alkylthio” refers to an alkyl radical attached to the remainder of the molecule by sulfur; this includes, but is not limited to, methylthio, ethylthio, and propylthio groups.

The term “cycloalkyl” refers to a cyclic group of from three to six carbon atoms; preferred cycloalkyl groups are cyclopentyl and cyclohexyl.

The term “aryl” refers to aromatic hydrocarbons such as phenyl, naphthyl, and the like which may be unsubstituted or substituted with radicals selected from alkyl, such as methyl or ethyl, alkoxy, such as methoxy or ethoxy, alkylthio, such as methylthio, halogen, hydroxy, nitro and trifluoromethyl.

The term “arylalkyl” is as defined above for alkyl and for aryl. Such groups include, but are not limited to, benzyl.

The following examples serve to illustrate certain embodiments of the invention, without limiting the invention to these particular embodiments. Those skilled in the art will recognize that the invention covers all alternatives, modifications and equivalents as may be included within the scope of the appended claims.

### EXAMPLE 1

### Preparation of O-Carbamoyl-(D)-phenylalaninol

In a dry 2L three-neck round bottomed flask equipped with a mechanical stirrer. thermometer and 250 mL addition funnel, 838 mL of dichloromethane was charged 5 followed by D-phenylalaninol (100 g, 0.66 mole) and sodium cyanate (85 g, 0.92 mole). The mixture was stirred in an ice-bath. The addition funnel was charged with methanesulfonic acid (222.3 g, 2.31 mol) which was slowly added to the reaction mixture so as to maintain the temperature below 5° C. The reaction mixture thickened after the completion of the addition. The ice-bath was removed and the reaction mixture was stirred until D-phenylalaninol was no longer detected by TLC analysis. To the reaction mixture. 80 grams of ice was added and the reaction mixture was cooled in an ice bath, and a 20% aqueous solution of sodium hydroxide was added at such a rate as to maintain the temperature below 5° C. until the pH of the aqueous phase was between 10 and 11 as measured by using pH paper. The mixture was transferred to a separatory funnel and the organic phase was separated. The aqueous phase was extracted with two 500 mL portions of dichloromethane, and the combined organic phase was washed with brine (350 mL) and dried over sodium sulfate (50 g) overnight. After removal of sodium sulfate by filtration, the organic phase was concentrated in vacuo to yield 115 g (89%) of the free base form of the desired product O-Carbamoyl-(D)-phenylalaninol as an oil.

O-Carbamoyl-(D)-phenylalaninol hydrochloride was prepared as follows. The crude reaction product O-Carbamoyl-(D)-phenylalaninol (115 g) was dissolved in 120 mL of isopropanol and was transferred to three-neck round bottom flask equipped with a mechanical stirrer. The mixture was chilled in an ice bath and the dropping funnel was charged with 100 mL of saturated HCl solution in isopropanol (6.5M). The HCl solution was slowly added to the free base solution so as to maintain the temperature below 5° C. During the addition, precipitation of the desired product in HCl form was observed. After the complete addition the mixture was stirred for another hour and 660 mL of acetone was added. The mixture was stirred for another hour and the white precipitate was collected by filtration. The product was washed thoroughly with ice-chilled isopropanol-acetone (1/3, v/v), and dried in vacuo. The product O-Carbamoyl-(D)-phenylalaninol hydrochloride weighed 110 gram (71.5%) and was a white solid.

### EXAMPLE 2

### Preparation of O-Carbamoyl-(D)-3.4-dichlorophenylalaninol

In a dry 2L three-neck round bottomed flask equipped with a mechanical stirrer. thermometer and 250 mL addition funnel. 75 mL of dichloromethane was charged 5 followed by (D)-3.4-dichlorophenylalaninol (4.00 g 0.018 mole) and sodium cyanate (1.87 g, 0.027 mole). The mixture was stirred in an ice-bath. The addition funnel was charged with methanesulfonic acid (4.37 g, 0.045 mol) which was slowly added to the reaction mixture so as to maintain the temperature below 5° C. The reaction mixture thickened after the completion of the addition. The ice-bath was removed and the reaction mixture was stirred until (D)-3.4-dichlorophenylalaninol was no longer detected by TLC analysis. A saturated aqueous solution of sodium bicarbonate was added to the reaction mixture at such a rate as to maintain the temperature below 5° C. until the pH of the aqueous phase was between 9 and 10. The mixture was transferred to a separatory funnel and the organic phase was separated. The aqueous phase was extracted with two 25 mL portions of dichloromethane, and the combined organic phase was washed with brine (30 mL) and dried over sodium sulfate (5 g) overnight. After removal of sodium sulfate by filtration, the organic phase was concentrated in vacuo to yield 4.38 g (91%) of the free base form of the desired product O-Carbamoyl-(D)-3.4-dichlorophenylalaninol as an oil.

O-Carbamoyl-(D)-3,4-dichlorophenylalaninol hydrochloride was prepared as follows. The crude reaction product O-Carbamoyl-(D)-3.4-dichlorophenylalaninol (3.27 g) was dissolved in 10 mL of tetrahydrofuran and was transferred to three-neck round bottom flask equipped with a mechanical stirrer. The mixture was chilled in an ice bath and the dropping funnel was charged with 13.7 mL of 1N HCl solution in ethyl ether (0.0137M). The HCl solution was slowly added to the free base solution so as to maintain the temperature below 5° C. During the addition, precipitation of the desired product in HCl form was observed. The white precipitate was collected by filtration. The product was washed thoroughly with ethyl ether, and dried in vacuo. The product O-Carbamoyl-(D)-3,4-dichlorophenylalaninol hydrochloride weighed 3.68 gram (99%) and was a white solid.

### EXAMPLE 3

### Preparation of O-Carbamoyl-(L)-3-oxymethyl-1.2.3.4-tetrahydroisoquinoline

(L)-3-hydroxymethyl-1.2.3.4-tetrahydroisoquinoline (194 g) was suspended in dichloromethane (1.5L) and the mixture was chilled in an ice-bath. To the resulting mixture, sodium cyanate (100.4 g) was added followed by dropwise addition of methanesulfonic acid (277.4 mL) so as to maintain the reaction temperature below 5° C. The addition took about 2 hours. The reaction mixture was stirred at room temperature until the reaction was complete. 1.5 Liters of deionized water was added to the reaction mixture. The aqueous phase was isolated and chilled in an ice-bath. The pH of the aqueous phase was adjusted to between 10 and 11 by adding 20% aqueous solution of sodium hydroxide. The resulting mixture was chilled in an ice-bath for about an hour and the product was filtered and washed with two 100 mL portions of deionized water. The product was dried under vacuum to yield 221.6 g (90.4%) of the desired product.

### EXAMPLE 4

### Large-Scale Preparation of O-Carbamoyl-(D)-phenylalaninol

Eighteen kilogram (18.0 kg) of D-phenylalaninol and 477.4 kg of dichloromethane were charged into a 300-gallon glass-lined reactor (Pfaudler, model R-01) blanketed with nitrogen. The solution was cooled to 4.8° C. Sodium cyanate (10.8 kg) was then added. To this mixture methanesulfonic acid (39.0 kg) was slowly charged over 2 hours and 42 minutes while maintaining the temperature below 5° C. After the addition was complete, the mixture was allowed to warm to 22.4° C. over 2 hours and 3 minutes, and agitated at ambient temperature for 16 hours and 50 minutes, at which time a sample was submitted to quality control for analysis by HPLC and the amount of D-phenylalaninol was less than 1.0%. The reactor contents were cooled to 4.1° C. and 100L of a 10% solution of sodium hydroxide (prepared by dissolving 12.0 kg sodium hydroxide in 108L water) was added while maintaining the reactor contents at less than 5° C. so that the pH was raised from pH 1.4 to pH 10.5. The two layers were separated. The upper aqueous was further extracted two times by dichloromethane (133.4 kg each), and the three organic layers were combined. The product containing dichloromethane was washed with 100L of a 1% solution of sodium hydroxide (prepared by dissolving 1.2 kg of sodium hydroxide in 108L of water), and analyzed by HPLC. The level of late eluting impurities was less than 0.3%. The organic layer was washed with 50L of a 10% brine solution (prepared from dissolving 5 kg sodium chloride in 50L water), then with water (50L), and dried by adding anhydrous sodium sulfate (19 kg) and allowing the mixture to stand for 18 hours. The sodium sulfate was removed by vacuum filtration on a 45 cm Nutch funnel (Baxter filter paper grade 615-20). The filter cake was washed with dichloromethane (25 kg), and the filtrate was concentrated to approximately 100L on a rotary evaporator at 25-30° C. The material was transferred to glass trays, dried in a vacuum oven at 40° C. until a constant weight was achieved.

### EXAMPLE 5

### Large-Scale Preparation of O-Carbamoyl-(L)-3-oxymethyl-1.2.3.4-tetrahydroisoquinoline

A 300-gallon reactor was charged with acetonitrile (236 kg) and THIC-alcohol (15 kg). The reaction mixture was cooled to less than 5° C. and methanesulfonic acid (39.9 kg) and sodium cyanate (17.8 kg) were added. The reaction mixture was allowed to warm to about 20° C. and held at this temperature for about 2 hours. HPLC analysis of the reaction mixture was performed to indicate that the reaction had gone to completion. The reaction mixture was diluted with toluene (104 kg) and cooled to less than 5° C. for 1 hour. The solid was isolated by filtration and the cake was washed with about 30L of toluene. The wet cake was added back to a 100-gallon reactor containing 10.1 kg of concentrated HCl in 150L of water. An in-process HPLC analysis showed that the reaction mixture contained no impurities greater than 1%. The reaction mixture was filtered to remove particulate matter. Then the upper toluene layer was removed and discarded. The aqueous layer was cooled to less than 5° C. and the pH adjusted to 10.5 by carefully adding 20% aqueous sodium hydroxide. The mixture was stirred for 1 hour then the solid was collected by filtration. The wet cake was slurry washed with water (50L) and refiltered. The product was dried in vacuo at 40° C. to yield 14.79 kg of product, which was found to be 98.77% pure by HPLC assay.

### EXAMPLE 6

### Large-Scale Preparation of Carbamic Acid 2-((4-fluorobenzoyl)piperidin-1-yl)-1-phenylethyl ester

A 100-gallon reactor was charged with dichloromethane (210.1 kg) and 2-(4-5 fluorobenzoyl)piperidin-1-yl)-1-phenylethanol (15.9 kg). The mixture was stirred at 100 rpm and cooled to 5° C.±5° C. Methanesulfonic acid (9.4 kg) was added to the solution over a twenty-minute period while maintaining the temperature below 10° C. Stirring was continued for 1 hour at 5° C.±5° C. Sodium cyanate was charged in five portions (total 6.4 kg) every five minutes while maintaining the temperature under 10° C. The reaction mixture was stirred for thirty minutes at this temperature, then stirred overnight at 25° C.±5° C. At one point, upon warming, the temperature of the reaction mixture briefly rose to 30.7° C. Another 0.7 kg of sodium cyanate and 1.1 kg of methanesulfonic acid were added to the reaction mixture and stirred at 25° C.±5° C. overnight. An in-process HPLC test indicated that the reaction had not gone to completion (<5% starting material). Thus, additional sodium cyanate (1.3 kg) and methanesulfonic acid (2.6 kg) were added to the reactor and stirred continuously for 8 hours. At this time the reaction mixture was found to contain only 3.2% starting material. The solid was collected by filtration. The filter cake was washed with two portions (23.0 kg. 22.5 kg) of dichloromethane. The wet cake was held overnight under a nitrogen atmosphere. The crude product was charged back to a 100-gallon reactor containing 140L of deionized water. The mixture was stirred at 90 rpm and cooled to 5° C.±5° C. A 50% solution of sodium hydroxide (7.6 kg) was added to the reactor while maintaining the temperature below 10° C. The mixture was stirred at this temperature for one hour then the solid was isolated by filtration. The filter cake was washed with 49L of deionized water. The solid was charged back into a reactor containing 52.5 kg of heptane. The mixture was stirred for 15 minutes then the solid was isolated by filtration. The solid was washed with heptane (2.3 kg) and then dried overnight in vacuo (27 mm) at 25° C.

The dried material (16.8 kg) was charged back to a reactor containing 464.1 kg of dichloromethane. The mixture was heated to reflux (40° C.) for one hour. The slurry was cooled to 34° C.±5° C. and passed through a Cuno Filter into a clean reactor. The filter was rinsed with two portions (22.3 kg each) of warm (31° C.) dichloromethane. The combined filtrate was reduced in volume to approximately 240L. The slurry was cooled to 3° C.±5° C. for 2 hours and the solid was then collected by filtration. The filter cake was washed with 29.5 kg of dichloromethane. The solid was dried in vacuo in a rotary cone drier at 28° C. for 46.5 hours. The product so obtained weighted 12.2 kg, representing a 67.9% yield.

It is understood that various other embodiments and modifications in the practice of the invention will be apparent to, and can be readily made by, those skilled in the art without departing from the scope of the invention described above. Accordingly, it is not intended that the scope of the claims appended hereto be limited to the exact description set forth above, but rather that the claims be construed as encompassing all of the features of patentable novelty which reside in the present invention, including all the features and embodiments which would be treated as equivalents thereof by those skilled in the art to which the invention pertains.

What is claimed is:

1. A process for preparing an O-carbamoyl aminoalcohol represented by Formula I 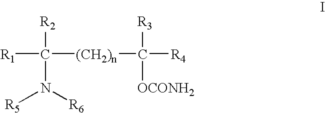
wherein:
n is an integer from 0 and 5;
R₁, R₂, R₃ and R₄ are individually selected from the group consisting of hydrogen, alkyl, cycloalkyl, substituted or unsubstituted aryl and arylalkyl wherein the aryl portion of which may be unsubstituted or substituted by (X′)ₘ, wherein m is an integer from 0 to 4 and X′ is selected from the group consisting of hydrogen, alkyl, alkoxy, alkylthio, halogen, hydroxy, nitro and trifluoromethyl;
R₅ and R₆ are individually selected from a group consisting of hydrogen, alkyl and arylalkyl wherein the aryl portion may be substituted or unsubstituted by (X′)ₘ, wherein m and X′ are as defined; or
R₁ and R₅ together with the carbon and nitrogen to which they are attached may form an unfused or fused heterocyclic ring having from 4 to 10 members;
the process comprising reacting an aminoalcohol represented by Formula II 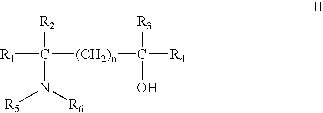
wherein n, R₁, R₂, R₃, R₄, R₅ and R₆ are as defined;
with a cyanate and an excess of an acid in an organic solvent medium.

2. A process according to claim 1, wherein the cyanate is an alkali cyanate or alkaline earth cyanate.

3. A process according to claim 2, wherein the cyanate is selected from the group consisting of sodium cyanate, potassium cyanate, ammonium cyanate, magnesium cyanate, and calcium cyanate.

4. A process according to claim 1, wherein the acid is selected from the group consisting of hydrochloric acid, sulfuric acid, phosphoric acid, acetic acid, halogenated acetic acids, arylsulfonic acids, alkylsulfonic acids and halogenated alkylsulfonic acids.

5. A process according to claim 1, wherein the organic solvent medium is selected from the group consisting of halogenated alkanes solvents, ethereal solvents, nitrile solvents, aromatic solvents, and mixtures thereof.

6. A process according to claim 1, wherein the cyanate is sodium cyanate and the acid is methanesulfonic acid.

7. A process according to claim 6, wherein the organic solvent medium is dichloromethane or acetonitrile.

8. A process according to claim 1, wherein the O-carbamoyl aminoalcohol is represented by Formula III 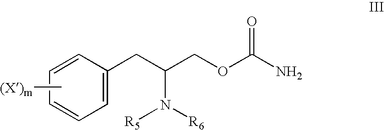
wherein X′, m, R₅ and R₆ are as defined;
the process comprising reacting an aminoalcohol represented by Formula IV 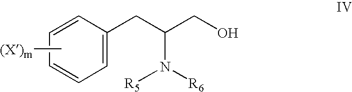
wherein X′, m, R₅ and R₆ are as defined:
with a cyanate and an excess of an acid in an organic solvent medium.

9. A process according to claim 1, wherein the O-carbamoyl aminoalcohol is represented by Formula V 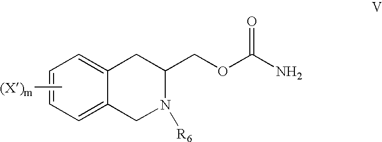
wherein X′, m, and R₆ are as defined:
the process comprising reacting an aminoalcohol represented by Formula VI 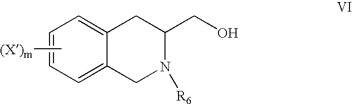
wherein X′, m, and R₆ are as defined;
with a cyanate and an excess of an acid in an organic solvent medium.

10. A process according to claim 1, wherein the O-carbamoyl aminoalcohol is represented by Formula VII 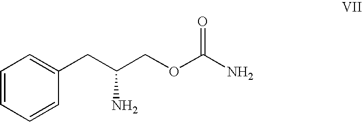
the process comprising reacting D-phenylalaninol represented by Formula VIII 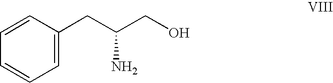
with a cyanate and an excess of an acid in an organic solvent medium.

11. A process according to claim 10, wherein the cyanate is selected from the group consisting of sodium cyanate, potassium cyanate, ammonium cyanate, magnesium cyanate, and calcium cyanate: the acid is selected from the group consisting of hydrochloric acid, acetic acid, trifluoroacetic acid, trichloroacetic acid, benzenesulfonic acid, toluenesulfonic acid, methanesulfonic acid, ethanesulfonic acid, and trifluoromethanesulfonic acid: and the organic solvent medium is selected from the group consisting of dichloromethane, chloroform, 1.2-dichloroethane, 1.1.1-trichloroethane, tetrahydrofuran, 1.2-dimethoxyethane, diethyl ether, acetonitrile, propionitrile, benzene, toluene, xylene, and mixtures thereof.

12. A process according to claim 10, wherein the cyanate is sodium cyanate and the acid is methanesulfonic acid.

13. A process according to claim 12, wherein the organic solvent medium is dichloromethane.

14. A process according to claim 1, wherein the O-carbamoyl aminoalcohol is O-carbamoyl-(L)-oxymethyl-1.2.3.4-tetrahydroisoquinoline represented by Formula IX 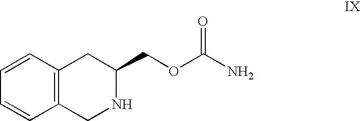
the process comprising reacting (L)-hydroxymethyl-1.2.3.4-tetrahydroisoquinoline represented by Formula X 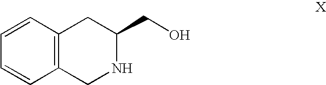
with a cyanate and an excess of an acid in an organic solvent medium.

15. A process according to claim 14, wherein the cyanate is selected from the group consisting of sodium cyanate, potassium cyanate, ammonium cyanate, magnesium cyanate, and calcium cyanate: the acid is selected from the group consisting of hydrochloric acid, acetic acid, trifluoroacetic acid, trichloroacetic acid, benzenesulfonic acid, toluenesulfonic acid, methanesulfonic acid, ethanesulfonic acid, and trifluoromethanesulfonic acid: and the organic solvent medium is selected from the group consisting of dichloromethane, chloroform, 1.2-dichloroethane, 1.1.1-trichloroethane, tetrahydrofuran, 1.2-dimethoxyethane, diethyl ether, acetonitrile, propionitrile, benzene, toluene, xylene, and mixtures thereof.

16. A process according to claim 14, wherein the cyanate is sodium cyanate and the acid is methanesulfonic acid.

17. A process according to claim 16, wherein the organic solvent medium is dichloromethane.

18. A process according to claim 16, wherein the organic solvent medium is acetonitrile.

19. A process according to claim 1, wherein the O-carbamoyl aminoalcohol is carbamic acid 2-((4-fluorobenzoyl)piperidin-1-yl)-1-phenylethyl ester represented by Formula XI: 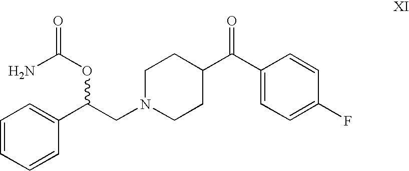
the process comprising reacting 2-(4-fluorobenzoyl)piperidin-1-yl)-1-phenylethanol represented by Formula XII 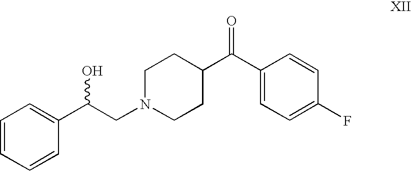
with a cyanate and an excess of an acid in an organic solvent medium.

20. A process according to claim 19, wherein the cyanate is selected from the group consisting of sodium cyanate, potassium cyanate, ammonium cyanate, magnesium cyanate, and calcium cyanate: the acid is selected from the group consisting of hydrochloric acid, acetic acid, trifluoroacetic acid, trichloroacetic acid, benzenesulfonic acid, toluenesulfonic acid, methanesulfonic acid, ethanesulfonic acid, and trifluoromethanesulfonic acid: and the organic solvent medium is selected from the group consisting of dichloromethane, chloroform, 1.2-dichloroethane, 1,1,1-trichloroethane, tetrahydrofuran, 1.2-dimethoxyethane, diethyl ether, acetonitrile, propionitrile, benzene, toluene, xylene, and mixtures thereof.

21. A process according to claim 19, wherein the cyanate is sodium cyanate and the acid is methanesulfonic acid.

22. A process according to claim 21, wherein the organic solvent medium is dichloromethane.

23. A process according to claim 1, wherein the amount of the acid is between about one to about ten molar equivalents in excess of the total number of amine groups in the aminoalcohol represented by Formula II.

24. A process according to claim 1, wherein the molar ratio of cyanate to aminoalcohol represented by Formula II is between about one to about ten.

25. A process according to claim 1, wherein the weight to volume ratio of the amount of the aminoalcohol represented by Formula II to the amount of the organic solvent medium is within the range of from about 1:3 to about 1:100.

26. A process according to claim 1, wherein the reaction is carried out at a temperature ranging from about −80° C. to about 80° C.

27. A process according to claim 25, wherein the reaction is carried out at a temperature ranging from about −10° C. to about 60° C.

28. A process according to claim 1, wherein the O-carbamoyl aminoalcohol represented by Formula I and aminoalcohol represented by Formula II are in the racemic form.

29. A process according to claim 1, wherein the O-carbamoyl aminoalcohol represented by Formula I and aminoalcohol represented by Formula II are in optically active form.

30. A process according to claim 1, wherein the O-carbamoyl aminoalcohol represented by Formula I and aminoalcohol represented by Formula II are in are in the S-form.

31. A process according to claim 1, wherein the O-carbamoyl aminoalcohol represented by Formula I and aminoalcohol represented by Formula II are in the R-form.
